(19) United States
(12) Patent Application Publication
(10) Pub. No.: US 2004/0249134 A1
(43) Pub. Date: Dec. 9, 2004

(54) Factor viii c2 domain variants

(76) Inventor: John S. Lollar, Decatur, GA (US)

Correspondence Address: Greenlee Winner & Sullivan, Suite 201, 5370 Manhanttan Circle, Boulder, CO, 80303, US

(21) Appl. No.: 10/491,464

(22) PCT Filed: Nov. 27, 2002
(86) PCT No.: PCT/US02/37884

Related U.S. Application Data
(60) Provisional application No. 60/334,569, filed on Nov. 30, 2001.

(51) Int. Cl. A61K 38/38

(52) U.S. Cl. 530/383

(57) ABSTRACT

Specific amino acid loci of human factor VIII interact with inhibitory antibodies of hemophilia patients after being treated with factor VIII. Modified factor VIII is disclosed in which the amino acid sequence is changed by a substitution at one or more of the specific loci. The modified factor VIII si useful for hemophiliacs, either to avoid or prevent the action of inhibitory antibodies.

### CROSS-REFERENCE TO RELATED APPLICATIONS

This application claims priority from U.S. Provisional Patent Application No. 60/334,569, filed Nov. 30, 2001.

### ACKNOWLEDGMENT OF FEDERAL RESEARCH SUPPORT

This invention was made, at least in part, with funding from the National Institutes of Health under contract No. F01-HL46215. Accordingly, the U.S. government may have certain rights in this invention.

### FIELD OF THE INVENTION

This invention relates generally to a modified mammalian factor VIII having amino acid substitutions which reduce its immunogenicity and/or antigenicity as compared to the proteins from which they were derived or other factor VIII preparations such as human factor VIII.

### BACKGROUND OF THE INVENTION

Blood clotting begins when platelets adhere to the cut wall of an injured blood vessel at a lesion site. Subsequently, in a cascade of enzymatically regulated reactions, soluble fibrinogen molecules are converted by the enzyme thrombin to insoluble strands of fibrin that hold the platelets together in a thrombus. At each step in the cascade, a protein precursor is converted to a protease that cleaves the next protein precursor in the series. Co-factors are required at most of the steps.

Factor VIII circulates as an inactive precursor in blood, bound tightly and non-covalently to von Willebrand factor. Factor VIII is proteolytically activated by thrombin or factor Xa, which dissociates it from von Willebrand factor and activates its procoagulant function in the cascade. In its active form, the protein factor VIIIa is a cofactor that increases the catalytic efficiency of factor IXa toward factor X activation by several orders of magnitude.

People with deficiencies in factor VIII or antibodies against factor VIII who are not treated with factor VIII suffer uncontrolled internal bleeding that may cause a range of serious symptoms, from inflammatory reactions in joints to early death. Severe hemophiliacs, who number about 10,000 in the United States, can be treated with infusion of human factor VIII, which will restore the blood's normal clotting ability if administered with sufficient frequency and concentration. The classical definition of factor VIII is that substance present in normal blood plasma that corrects the clotting defect in plasma derived from individuals with hemophilia A.

The development of antibodies (“inhibitors” or “inhibitory antibodies”) that inhibit the activity of factor VIII is a serious complication in the management of patients with hemophilia. Autoantibodies develop in approximately 20% of patients with hemophilia A in response to therapeutic infusions of factor VIII. In previously untreated patients with hemophilia A who develop inhibitors, the inhibitors usually develops within one year of treatment. Additionally, autoantibodies that inactivate factor VIII occasionally develop in individuals with previously normal factor VIII levels. Inhibitory antibodies (inhibitors) to factor VIII (fVIII) either develop as alloantibodies in hemophilia A patients following fVIII infusions or as autoantibodies in nonhemophiliacs [Hoyer, L. W. and D. Scandella (1994) Semin.Hematol. 31:1-5]. Antibodies to epitopes in the A2, ap-A3, and C2 domains within the A1-A2-B-ap-A3-C1-C2 fVIII molecule are responsible for all anticoagulant activity in most inhibitor plasmas [Prescott, R. el al., (1997) Blood 89:3663-3671; Barrow, R. T. et al., (2000) Blood 95:557-561]. The 18-kDa C2 domain, defined as residues Ser2173- Tyr2332 in single chain human fVIII, contains a phospholipid membrane-binding site that is necessary for the normal procoagulant function of fVIII. Human anti-fVIII antibodies specific for the C2 domain inhibit this interaction [Arai, M. etal., (1989) J. Clin. Invest. 83:1978-1984]. Consistent with this, phospholipid protects fVIII from inactivation by fVIII inhibitors [Arai et al., supra; Barrowdliffe, T. W. et al., (1983) J. Lab. Clin. Med. 101:34-43]. The C2 domain also contains part of the von Willebrand factor (vWf) binding site (Saenko, E. L. et al., (1994) J. Biol. Chem. 269:11601-11605; Saenko, E. L. and Scandella, D. (1997) J. Biol. Chem. 272:18007-180141. Some inhibitors may act by interfering with this interaction [Shima, M. et al., (1995) Br. J. Haematol. 91:714-72 1; Saenko, E. L. et al., (1996)J. Biol. Chem. 271:27424-2743 1; Gilles, J. G. et al., (1999) Thromb. Haemost. 82:40-45].

Patients with hemophilia A can be managed by increasing the dose of factor VIII provided the inhibitor titer is low enough. However, often the inhibitor titer is so high that it cannot be overcome by factor VIII administration. An alternative strategy is to bypass the need for factor VIII during normal hemostasis using factor IX complex preparations (for example, KONYNE®, Proplex®) or recombinant human factor VIIIa. Additionally, since porcine factor VIII usually has substantially less reactivity with inhibitors than human factor VIII, a partially purified porcine factor VIII preparation (HYATE:C®) is used. Many patients who have developed inhibitory antibodies to human factor VIII have been successfully treated with porcine factor VIII and have tolerated such treatment for long periods of time. However,administration of porcine factor VIII is not a complete solution because inhibitors may develop to porcine factor VIII after one or more infusions.

Several preparations of human plasma-derived factor VIII of varying degrees of purity are available commercially for the treatment of hemophilia A. These include a partially-purified factor VIII derived from the pooled blood of many donors that is heat- and detergent-treated for viruses but contain a significant level of antigenic proteins; a monoclonal antibody-purified factor VIII that has lower levels of antigenic impurities and viral contamination; and recombinant human factor VIII, clinical trials for which are underway. Unfortunately, human factor VIII is unstable at physiologic concentrations and pH, is present in blood at an extremely low concentration (0.2 μg/ml plasma), and has low specific clotting activity.

Hemophiliacs require daily replacement of factor VIII to prevent bleeding and the resulting deforming hemophilic arthropathy. However, supplies have been inadequate and problems in therapeutic use occur due to difficulty in isolation and purification, immunogenicity, and the risk of contamination by viruses such as HIV, hepatitis and the like. The use of recombinant human factor VIII or partially-purified porcine factor VIII will not resolve all the problems.

The problems associated with the commonly used, commercially available, plasma-derived factor VIII have stimulated significant interest in the development of a better factor VIII product. There is a need for a high specific activity factor VIII molecule so that adequate clotting activity can be delivered in a smaller dose; a factor VIII molecule that is stable at a selected pH and physiologic concentration; a factor VIII molecule that is less immunogenic; and a factor VIII molecule that is not inhibited in patients who have already developed antibodies to human factor

U.S. Pat. No. 6,180,371 to Lollar describes amino acid substitutions in the A2 domain of human factor VIII which alter the antigenicity of the resulting factor VIII molecules. U.S. Pat. No. 5,859,204 to Lollar discloses the site specific replacement of amino acids in the 484-509 region of human factor VIII. More specifically, the '204 patent teaches modified factor VIII with amino acid substitutions at positions 485, 487, 488, 489, 492, 495, 501 or 508 relative to the human protein. U.S. Pat. No. 5,888,974 to Lollar et al. discloses hybrid procoagulant factor VIII produced by the isolation and recombination of human and other non-human factor VIII subunits or domains. U.S. Pat. No. 5,744,446 to Lollar et al. describes hybrid factor VIII having amino acid substitutions in the A2 domain. U.S. Pat. No. 5,663,060 to Lollar et al. describes hybrid factor VIII having combinations of non-human and human heavy and light chain subunits. U.S. Pat. No. 5,583,209 describes nucleic acids encoding the hybrid factor VIII molecules in the '060 patent. U.S. Pat. No. 5,364,771 describes purified hybrid factor VIII made of human and porcine combinations of the heavy and light subunits. Also disclosed is human factor VIII with porcine A2 domain substituted for the human A2 domain.

U.S. Pat. Nos. 6,180,371; 5,888,974; 5,859,204; 5,744,446; 5,663,060; 5,583,209; and 5,364,771 (all of which are incorporated herein by reference) do not disclose substitutions or suggest specific amino acid substitutions in the C2 domain of factor VIII which reduce antigenicity or immunogenicity as compared to wild-type factor VIII or the corresponding recombinant factor VIII.

It is therefore an object of the present invention to provide a modified factor VIII that corrects hemophilia in a patient deficient in factor VIII or having inhibitory antibodies to the C2 domain of factor VIII.

It is a further object of the present invention to provide methods for treatment of hemophiliacs.

It is still another object of the present invention to provide a factor VIII that is stable at a selected pH and physiologic concentration.

It is yet another object of the present invention to provide a factor VIII that has greater coagulant activity than human factor VIII.

### SUMMARY OF THE INVENTION

The present invention generally relates to recombinant modified factor VIII. The compositions of the invention provide isolated, purified recombinant modified factor VIII molecules with coagulant activity wherein the recombinant factor VIII has amino acid substitutions in the C2 domain which reduce antigenicity as compared to normal human factor VIII or other factor VIII having a normal human factor VIII C2 domain. DNA sequences encoding the compositions of the invention as well as methods of producing the modified recombinant factor VIII are also provided. Methods of treating patients in need of treatment with factor VIII are also within the scope of this invention.

A first embodiment of the invention provides compositions having recombinant mammalian factor VIII with amino acid substitution(s) in the C2 domain. The amino acid substitution(s) in the C2 domain of the modified recombinant factor VIII reduce the anticoagulant activity of inhibitory antibodies as compared to normal human factor VIII or factor VIII having a normal human factor VIII C2 domain. The compositions of this embodiment have coagulant activity and reduced binding to inhibitory antibodies directed against the C2 domain.

In one aspect of this embodiment, the compositions relate to recombinant mammalian factor VIII having at least one amino acid substitution in the C2 domain at positions corresponding to human factor VIII at R2215, W2313, R2220, R2320, Y2195, F2196 and F2290. The compositions of this embodiment can be a single mutant, a double mutant, a triple mutant, or other multiple mutants. Examples of amino acid substitutions of the invention include, but are not limited to, R2215A, R2215K, W2313A, W2313F, R2220A, R2220K, R2320A, R2320K, Y2195H, Y2195A, F2196L, F2196A, F2290S and F2290A, all of which are referenced to the human factor VIII numbering system wherein amino acid number 1 is the amino terminal alanine of mature factor VIII. Substitutions in either recombinant porcine or human factor VIII are preferred. Preferred amino acid substitutions include those which are immunoreactivity reducing. Substitutions at positions 2220, 2196, and 2215 are preferred.

A second embodiment of the invention provides novel hybrid factor VIII compositions having recombinant factor VIII with amino acid substitution(s) in the C2 domain. The novel compositions of this embodiment are constructed by preparing hybrid factor VIII with amino acid substitutions in the C2 domain. The other domains of factor VIII may be derived from a variety of mammals such as human, mouse, pig, rat, and canine and so on. The novel compositions of this embodiment have coagulant activity and reduced binding to inhibitory antibodies. Examples of amino acid substitutions of the invention include, but are not limited to, R2215A, R2215K, W2313A, W2313F, R2220A, R2220K, R2320A, R2320K, Y2195H, Y2195A, F2 96L, F2196A, F2290S and F2290A, all of which are referenced to the human factor VIII numbering system wherein amino acid number 1 is the amino terminal alanine of mature factor VIII. Substitutions in either recombinant porcine or human factor VIII are preferred. Preferred amino acid substitutions include those which are immunoreactivity reducing. Substitutions at positions 2220, 2196, and 2215 are preferred.

Another embodiment of the invention provides DNA sequences comprising coding sequences for the novel compositions of the invention. Yet another embodiment of the invention provides methods of producing the novel compositions of the invention.

The invention also provides a method for reducing the immunogenicity of factor VIII molecules as well as recombinant factor VIII with reduced immunogenicity produced by the method. In particular, modified recombinant factor VIII molecule and methods of making such molecules with reduced immunogenicity that have substitutions in the C2 domain are described.

Also provided are pharmaceutical compositions and methods for treating patients having factor VIII deficiency comprising administering recombinant modified factor VIII and hybrid version thereof.

### BRIEF DESCRIPTION OF THE DRAWINGS

FIGS. 1A-1H taken together provide an aligned sequence comparison of the human, pig and mouse factor VIII amino acid sequences.

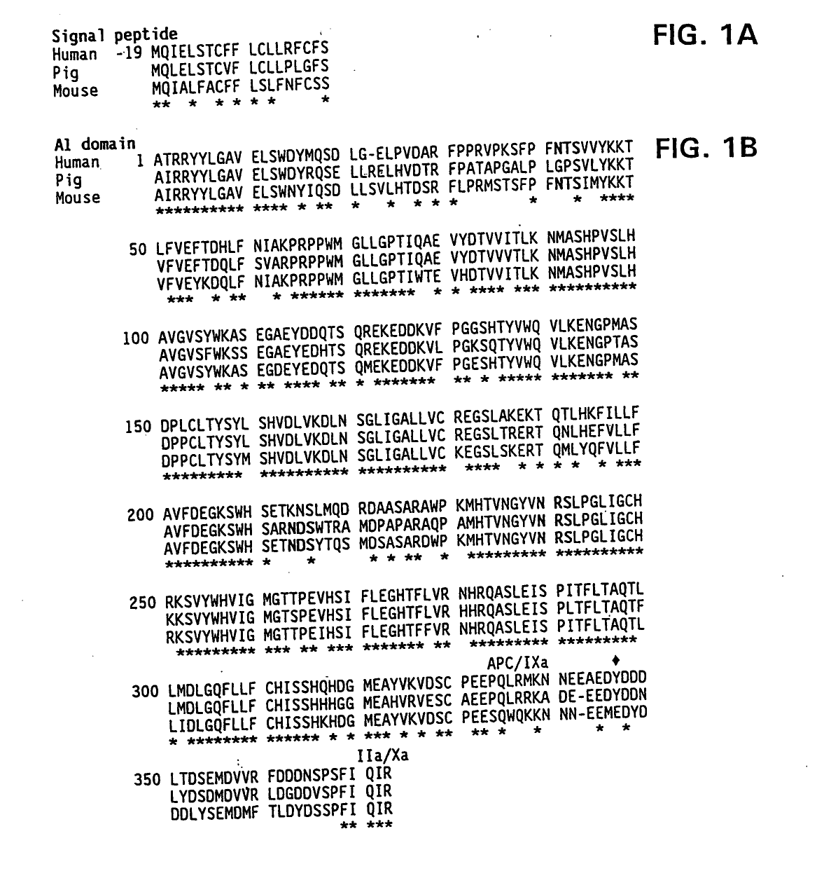

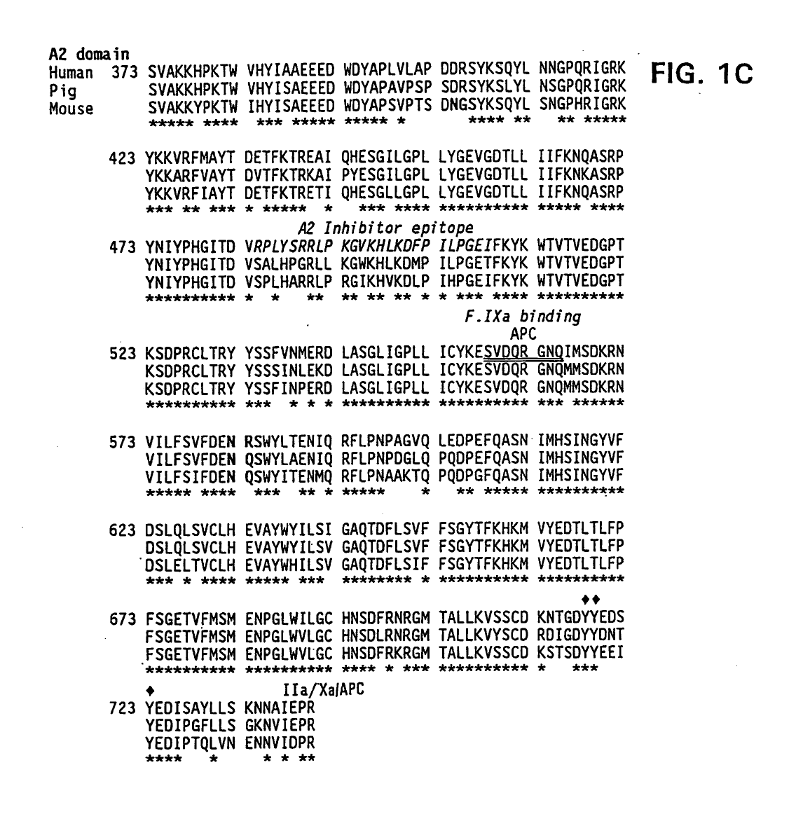

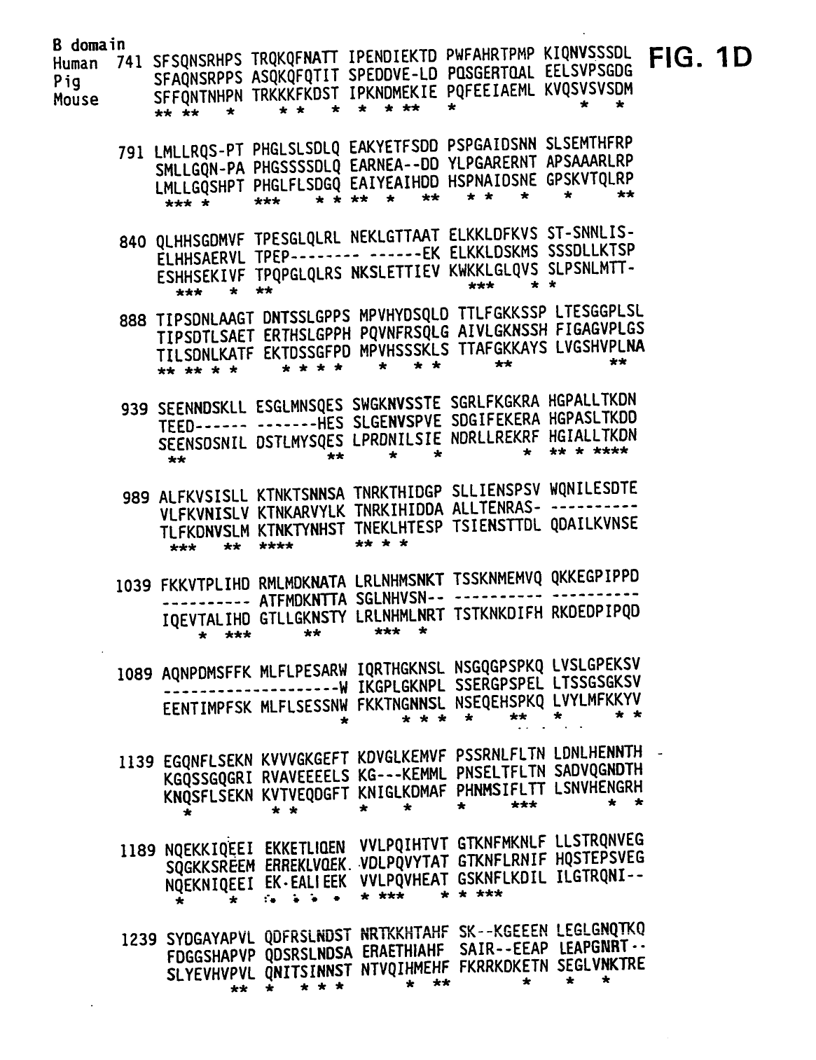

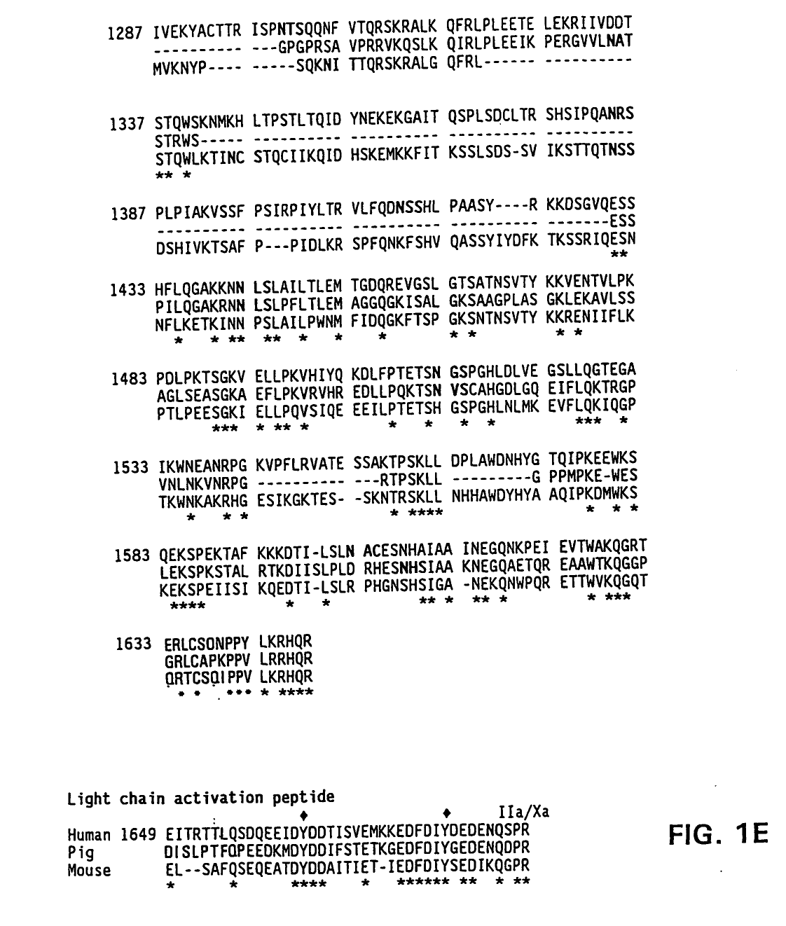

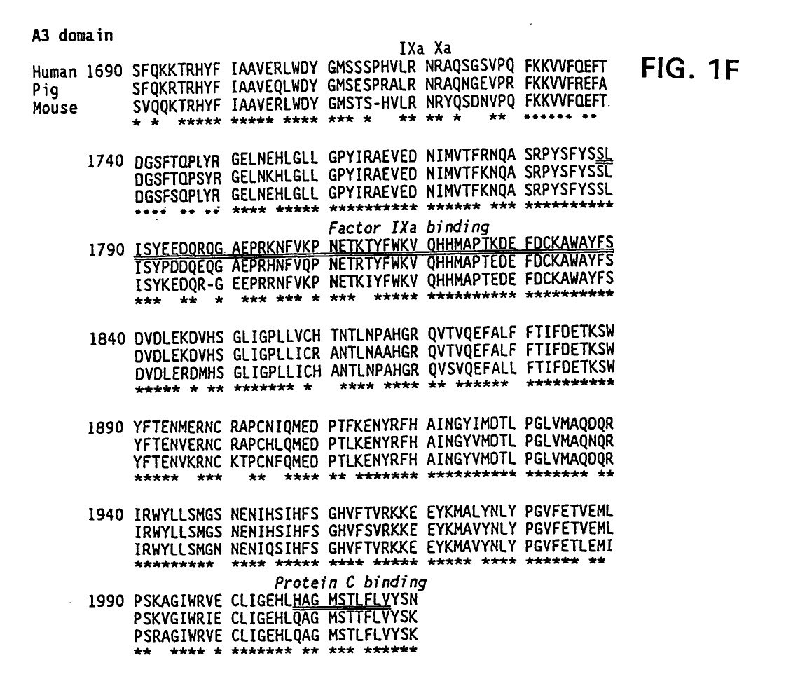

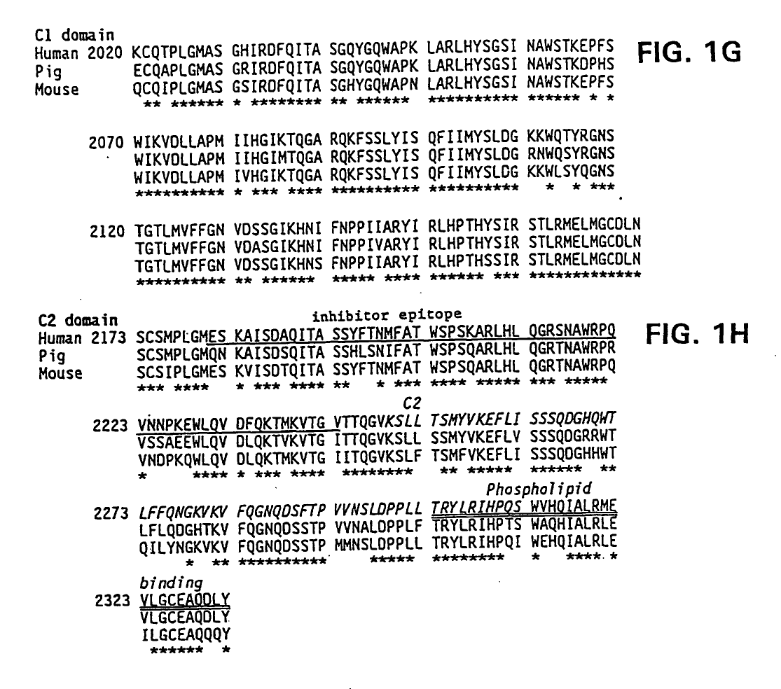

### DETAILED DESCRIPTION OF THE INVENTION

The present invention generally relates to recombinant modified factor VIII. The composition of the invention provides isolated, purified recombinant modified factor VIII molecules with coagulant activity. It was discovered that mutations in the C2 domain of factor VIII in amino acid residues identified in a recently available x-ray structure, reduced the binding of inhibitory antibodies of the mutants as compared to the normal human factor VIII or factor VIII having a normal human factor VIII C2 domain. Thus, the compositions of the invention provide recombinant factor VIII with amino acid substitutions in the C2 domain which reduce antigenicity as compared to normal human factor VIII or factor VIII having a normal human factor VIII C2 domain. Furthermore, the invention also provides recombinant factor VIII with amino acid substitutions in the C2 domain which reduce antigenicity as compared to other available factor VIII preparations. The invention also provides recombinant factor VIII with immunoreactivity reducing amino acid substitutions in the C2 domain. Related embodiments of the invention provide for methods of treating patients in need of factor VIII treatment, methods of producing the novel recombinant factor VIII compositions of the invention, DNA sequences encoding the novel recombinant factor VIII proteins, and pharmaceutical compositions comprising the novel factor VIII proteins.

The present invention further provides active recombinant hybrid factor VIII molecules or fragments thereof, the nucleic acid sequences encoding these hybrids, methods of preparing and isolating them, and methods for characterizing them. These hybrids can be human/animal, animal/animal, porcine/human or other such hybrid factor VIII molecules, and further have at least one specific amino acid sequence in the C2 domain including one or more unique amino acids of the factor VIII of one species substituted for the corresponding amino acid sequence (or amino acid) of the factor VIII of the other species; or have at least one sequence in the C2 domain including one or more amino acids having no known sequence identity to factor VIII substituted for specific amino acid sequence in human, animal, porcine or hybrid factor VIII. The resulting recombinant hybrid factor VIII has reduced or no immunoreactivity to factor VIII inhibitory antibodies, compared to proteins from which they were derived.

A “corresponding” nucleic acid or amino acid or sequence of either, as used herein, is one present at a site in a factor VIII molecule or fragment thereof that has the same structure and/or function as a site in the factor VIII molecule of another species, although the nucleic acid or amino acid number may not be identical. A DNA sequence “corresponding to” another factor VIII sequence substantially corresponds to such sequence, and hybridizes to the sequence of the designated SEQ ID NO. under stringent conditions. A DNA sequence “corresponding to” another factor VIII sequence also includes a sequence that results in the expression of a factor VIII or fragment thereof and would hybridize to the designated SEQ ID NO. but for the redundancy of the genetic code.

A “unique” amino acid residue or sequence, as used herein, refers to an amino acid sequence or residue in the factor VIII molecule of one species that is different from the homologous residue or sequence in the factor VIII molecule of another species. “Specific activity,” as used herein, refers to the activity that will correct the coagulation defect of human factor VIII deficient plasma. Specific activity is measured in units of clotting activity per milligram total factor VIII protein in a standard assay in which the clotting time of human factor VIII deficient plasma is compared to that of normal human plasma. One unit of factor VIII activity is the activity present in one milliliter of normal human plasma. In the assay, the shorter the time for clot formation, the greater the activity of the factor VIII being assayed. Porcine factor VIII has coagulation activity in a human factor VIII assay.

“Expression” refers to the set of processes that occur whereby genetic information is utilized to yield a product. A DNA encoding the amino acid sequence of porcine factor VIII can be “expressed” within a mammalian host cell to yield porcine factor VIII protein. The materials, genetic structures, host cells and conditions which permit expression of a given DNA sequence to occur are well-known in the art and can be manipulated to affect the time and amount of expression, as well as the intra- or extra-cellular location of the expressed protein. For example, by including DNA encoding a signal peptide at the 5′ end of the DNA encoding porcine factor VIII (the 5′ end being, by convention, that end encoding the NH₂ terminus of the protein) the expressed protein becomes exported from the interior of the host cell into the culture medium. Providing a signal peptide coding DNA in combination with the porcine factor VIII coding DNA is advantageous because the expressed factor VIII is exported into the culture medium which simplifies the process of purification. A preferred signal peptide is a mammalian factor VIII signal peptide.

Factor VIII is synthesized as an approximately 300 kDa single chain protein with internal sequence homology that defines the “domain” sequence NH₂-A 1-A2-B-A3-C1-C2-COOH. In a factor VIII molecule, a “domain”, as used herein, is a continuous sequence of amino acids that is defined by internal amino acid sequence identity and sites of proteolytic cleavage by thrombin. Unless otherwise specified, factor VIII domains include the following amino acid residues, when the sequences are aligned with the human amino acid sequence A1, residues Ala1-Arg372; A2, residues Ser373-Arg740; B, residues Ser741-Arg1648; A3, residues Ser1690-1le2032; C1, residues Arg2033-Asn2172; C2, residues Ser2173-Tyr2332. The A3-C1-C2 sequence includes residues Ser1690-Tyr2332. The remaining segment, residues Glu1649-Arg1689, is usually referred to as the factor VIII light chain activation peptide. Factor VIII is proteolytically activated by thrombin or factor Xa, which dissociates it from von Willebrand factor, forming factor VIIIa, which has procoagulant function. The biological function of factor VIIIa is to increase the catalytic efficiency of factor IXa toward factor X activation by several orders of magnitude. Thrombin-activated factor VIIIa is a 160 kDa A1/A2/A3-C1-C2 heterotrimer that forms a complex with factor IXa and factor X on the surface of platelets or monocytes. A “partial domain” as used herein is a continuous sequence of amino acids forming part of a domain.

“Subunits” of human or animal factor VIII, as used herein, are the heavy and light chains of the protein. The heavy chain of factor VIII contains three domains, A1, A2, and B. The light chain of factor VIII also contains three domains, A3, C1, and C2.

The terms “epitope,” “antigenic site,” and “antigenic determinant,” as used herein, are used synonymously and are defined as a portion of the human, or animal factor VIII or fragment there of that is specifically recognized by an antibody. It can consist of anynumberofamino acid residues, and it can be dependent upon the primary, secondary, or tertiary structure of the protein.

The term “immunogenic site,” as used herein, is defined as a region of the human or animal factor VIII, or fragment thereof, that specifically elicits the production of antibody to the factor VIII, or fragment, in a human or animal, as measured by routine protocols, such as immunoassay, e.g. ELISA, or the Bethesda assay, described herein. It can consist of any number of amino acid residues, and it can be dependent upon the primary, secondary, or tertiary structure of the protein. In some embodiments, the hybrid or hybrid equivalent factor VIII or fragment thereof is nonimmunogenic or less immunogenic in an animal or human than human or porcine factor VIII. “Factor VIII deficiency,” as used herein, includes deficiency in clotting activity caused by production of defective factor VIII, by inadequate or no production of factor VIII, or by partial or total inhibition of factor VIII by inhibitors. Hemophilia A is a type of factor VIII deficiency resulting from a defect in an X-linked gene and the absence or deficiency of the factor VIII protein it encodes.

As used herein, “diagnostic assays” include assays that in some manner utilize the antigen-antibody interaction to detect and/or quantify the amount of a particular molecule that is present in a test sample to assist in the selection of medical therapies. There are many such assays known to those of skill in the art. As used herein, human, porcine or modified porcine factor VIII DNA or fragment thereof and protein expressed therefrom, in whole or in part, can be substituted for the corresponding reagents in the otherwise known assays, whereby the modified assays may be used to detect and/or quantify antibodies to factor VIII. It is the use of these reagents, the factor VIII DNA or fragment thereof or protein expressed therefrom, that permits modification of known assays for detection of antibodies to human or animal factor VIII. Such assays include, but are not limited to ELISAs, immunodiffusion assays, and immunoblots. Suitable methods for practicing any of these assays are known to those of skill in the art. As used herein, the factor VIII or fragment thereof that includes at least one epitope of the protein can be used as the diagnostic reagent. Examples of other assays in which human, porcine or modified porcine factor VIII or fragment thereof can be used include the Bethesda assay and anticoagulation assays.

The term “DNA encoding a protein, such as porcine factor VIII” means a polydeoxynucleic acid whose nucleotide sequence embodies coding information to a host cell for the amino acid sequence of the protein, e.g. porcine factor VIII, according to the known relationships of the genetic code.

The “expression product” of a DNA encoding a human or animal factor VIII or a modified factor VIII is the product obtained from expression of the referenced DNA in a suitable host cell, including such features of pre- or post-translational modification of protein encoded by the referenced DNA, including but not limited to glycosylation, proteolytic cleavage and the like. It is known in the art that such modifications can occur and can differ somewhat depending upon host cell type and other factors, and can result in molecular isoforms of the product, with retention of procoagulant activity. See, e.g. Lind, P. et al. Eur. J. Biochem. 232:1927 (1995), incorporated herein by reference.

An “expression vector” is a DNA element, often of circular structure, having the ability to replicate autonomously in a desired host cell, or to integrate into a host cell genome and also possessing certain well-known features which permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art. The term “expression vector” is used to denote both a vector having a DNA coding sequence to be expressed inserted within its sequence, and a vector having the requisite expression control elements so arranged with respect to an insertion site that it can serve to express any coding DNA inserted into the site, all as well-known in the art. Thus, for example, a vector lacking a promoter can become an expression vector by the insertion of a promoter combined with a coding DNA.

“Immunoreactivity reducing” amino acids are defined herein as those amino acids that are minor contributors, if at all, to the binding energy of an antibody-antigen pair. Non-limiting examples of some amino acids known to be immunoreactivity-reducing include alanine, methionine, leucine, serine, and glycine. It will be understood that reduction of immunoreactivity achievable by a given substitution in a given antibody-antigen pair can also occur by substitution of amino acid other than those listed above if they affect protein conformation, eptope accessibility and the like.

Discovery of Mutations in Factor VIII Which Reduce Binding of Inhibitory Antibodies The fVIII C2 domain, consisting of amino acid residues 2173-2332, contains a major antigenic site or sites for most inhibitory antibodies in patients with hemophilia A or acquired hemophilia. The inhibitory action of these antibodies primarily appears to be due to inhibition of binding of fVIII to procoagulant phospholipid membranes. The X-ray structure of the human fVIII C2 domain reveals a putative hydrophobic phospholipid membrane-binding site consisting of loops containing M2199/F2200 and L2251/L2252 [Barrow, R. T., et al.(2001) Blood97:169-174]. These loops participate in binding inhibitory anti-C2 antibodies as judged by the reduction in antigenicity observed when they are substituted by homologous porcine, murine or canine residues. Identification of additional antigenic residues was accomplished by mutating seven surface-exposed sites around the membrane-binding site to create the following constructs: Y2195H, Y2195A, F2196L, F2196A, R2215K, R2215A, R2220K, R2220A, F2290S, F2290A, W2313F, W2313A, R2320K and R2320A. The mutants were expressed in baby hamster kidney cells. W2313A and R2320K yielded low level expression and were not evaluated further. To facilitate screening these mutants, 44 patient inhibitor plasmas were tested for C2 specificity using a recombinant human/porcine fVIII molecule, HP20, which contains the porcine C2 domain in place of the corresponding human domain. The cross-reactivity of 11 plasmas toward HP20 was as low or nearly as low as recombinant B domainless porcine fVIII, indicating that they are C2-specific. Of these plasmas, 8 were evaluated with respect to the C2 mutants using the Bethesda assay. Mutations at R2215, R2220, and F2196, but not W2313, R2320, F2290 or Y2195 produced lower antigenicity than wild type fVIII with respect to 2 inhibitor plasmas (DR and JF). The remaining six inhibitor plasmas did not demonstrate a reduction in Bethesda titer toward any of the mutants, indicating that they do not recognize amino acids R2215, R2220, F2196, W2313, R2320, F2290 or Y2195.

In conclusion, residues R2215, R2220, and F2196 contribute to the binding of fVIII to inhibitory antibodies. The data demonstrate that amino acid residues outside the membrane binding loops, can contribute to antibody binding. The data disclosed herein indicate that substitution of immunoreactivity reducing amino acids in such residues can reduce inhibition by inhibitory antibodies specific to the C2 domain of factor VIII. Substitution of immunoreactivity reducing amino acids at residues outside of the membrane binding loops with similar substitutions within the membrane binding loops, e.g., positions 2199,2220,2251, and 2252 can be expected to further reduce the inhibition of certain inhibitory antibodies reactive with the C2 domain.

Mutations were made in a B domainless form of human fVIII designated HSQ [Lind, P.,K. et al. (1995) Eur. J. Biochem. 232:19-27] by splicing-by-overlap extension mutagenesis as described previously [Lubin, I. M., etal.(1997)J. Biol. Chem. 272:30191-30195]. The following mutations, with corresponding nucleotide changes, were made:

| | |
|---|---|
| R2215A | AGG to GCC |
| R2215K | AGG to AAG |
| W2313A | TGG to GCC |
| W2313F | TGG to TTC |
| R2220A | AGA to GCC |
| R2220K | AGA to AAG |
| R2320A | AGG to GCC |
| R2320K | AGG to AAG |
| Y2195H | TAC to CAC |
| Y2195A | TAC to GCC |
| F2196L | TTT to CTG |
| F2196A | TTT to GCC |
| F2290S | TTC to TCT |
| F2290A | TTC to GCT |

### General Description of Methods

U.S. Pat. No. 5,364,771 described the discovery of hybrid human/porcine factor VIII molecules having coagulant activity, in which elements of the factor VIII molecule of human or pig are substituted for corresponding elements of the factor VIII molecule of the other species. U.S. Pat. No. 5,663,060 describes procoagulant hybrid human/animal and hybrid equivalent factor VIII molecules, in which elements of the factor VIII molecule of one species are substituted for corresponding elements of the factor VIII molecule of the other species.

Since current information indicates that the B domain has no inhibitory epitope and has no known effect on factor VIII function, in some embodiments the B domain is wholly or partially deleted in the active hybrid or hybrid equivalent factor VIII molecules or fragments thereof (“B(−) factor VIII”) prepared by any of the methods described herein.

The human factor VIII gene was isolated and expressed in mammalian cells, as reported by Toole, J. J. et al. (1984) Nature 312:342-347 (Genetics Institute); Gitschier, J. et al.(1984) Nature 312:326-330 (Genentech); Wood, W. I. et al. (1984) Nature 312:330-337 (Genentech); Vehar, G. A. etal. (1984) Nature 312:337-342 (Genentech); WO 87/04187; WO 88/08035; WO 88/03558; U.S. Pat. No. 4,757,006, and the amino acid sequence was deduced from cDNA. U.S. Pat. No. 4,965,199 to Capon et al. discloses a recombinant DNA method for producing factor VIII in mammalian host cells and purification of human factor VIII. Human factor VIII expression in CHO (Chinese hamster ovary) cells and BHKC (baby hamster kidney cells) has been reported. Human factor VIII has been modified to delete part or all of the B domain (U.S. Pat. No.4,868,112), and replacement of the human factor VIII B domain with the human factor V B domain has been attempted (U.S. Pat. No. 5,004,803).

Porcine factor VIII has been isolated from plasma [Fass, D. N. et al. (1982) Blood 59:594]. Partial amino acid sequence of porcine factor VIII corresponding to portions of the N-terminal light chain sequence having homology to ceruloplasmin and coagulation factor V was described by Church et al. (1984) Proc. Natl. Acad. Sci. USA 81:6934. Toole, J. J. et al. (1984) Nature 312:342-347 described the partial sequencing of the N-terminal end of four amino acid fragments of porcine factor VIII but did not characterize the fragments as to their positions in the factor VIII molecule. The amino acid sequence of the B and part of the A2 domains of porcine factor VIII were reported by Toole, J. J. et al. (1986) Proc. Natl. Acad. Sci, USA 83:5939-5942. The cDNA sequence encoding the complete A2 domain of porcine factor VIII and predicted amino acid sequence and hybrid human/porcine factor VIII having substitutions of all domains, all subunits, and specific amino acid sequences were disclosed in U.S. Pat. No. 5,364,771 entitled “Hybrid Human/Porcine factor VIII” issued on Nov. 15, 1994, and in WO 93/20093 published Oct. 14, 1993. The cDNA sequence encoding the A2 domain of porcine factor VIII corresponds to residues 373-740 in mature human factor VIII. More recently, the nucleotide and corresponding amino acid sequences of part of the A1 domain lacking the first 198 amino acids and of the A2 domain of porcine factor VIII were reported in WO 94/11503, published May 26, 1994. The entire nucleotide sequence encoding porcine factor VIII, including the complete A1 domain, activation peptide, A3, C1 and C2 domains, as well as the encoded amino acid sequence, was finally obtained by Lollar, as disclosed in U.S. Pat. 5,859,204, issued Jan. 12, 1999, and in WO 97/49725, published Dec. 31, 1997, both incorporated herein by reference.

Both porcine and human factor VIII are isolated from plasma as a two subunit protein. The subunits, known as the heavy chain and light chain, are held together by a non-covalent bond that requires calcium or other divalent metal ions. The heavy chain of factor VIII contains three domains, A1, A2, and B, which are linked covalently. The light chain of factor VIII also contains three domains, designated A3, C1, and C2. The B domain has no known biological function and can be removed, or partially removed from the molecule proteolytically or by recombinant DNA technology methods without significant alteration in any measurable parameter of factor VIII. Human recombinant factor VIII has a similar structure and function to plasma-derived factor VIII, though it is not glycosylated unless expressed in mammalian cells.

Both human and porcine activated factor VIII (“factor VIIIa”) have three subunits due to cleavage of the heavy chain between the A1 and A2 domains. This structure is designated A1/A2/A3-C1-C2. Human factor VIIIa is not stable under the conditions that stabilize porcine factor VIIIa, presumably because of the weaker association of the A2 subunit of human factor VIIIa. Dissociation of the A2 subunit of human and porcine factor VIIIa is associated with loss of activity in the factor Villa molecule. Yakhymv, A. et al. (1997) Blood 90:Suppl. 1, Abstract #126, reported binding of A2 domain by low density lipoprotein receptor-related protein, suggesting that cellular uptake of A2 mediated by such binding acts to down-regulate factor VIII activity.

Expression of “B-domainless factor VIII” is enhanced by including portions of the B-domain. The inclusion of those parts of the B domain designated “SQ” [Lind, P. et al. (1995) supra] was reported to result in favorable expression. “SQ” constructs lack all of the human B domain except for 5 amino acids of the B domain N-terminus and 9 amino acids of the B domain C-terminus. POL1212 constructs refer to cDNA encoding porcine factor VIII lacking most of the B domain but containing DNA sequence encoding a 24 amino acid linker between the A2 and ap domains as disclosed in U.S. Ser. No. 09/523,656 filed Mar. 10^{th} 2000, which is hereby incorporated by reference in its entirety.

The purified modified factor VIII or fragment thereof can be assayed for immunoreactivity and coagulation activity by standard assays including, for example, the plasma-free factor VIII assay, the one-stage clotting assay, and the enzyme-linked immunosorbent assay using purified recombinant human factor VIII as a standard.

Other vectors, including both plasmid and eukaryotic viral vectors, may be used to express a recombinant gene construct in eukaryotic cells depending on the preference and judgment of the skilled practitioner [see, for example, Chapter 16 in Sambrook et al. “Molecular Cloning” Cold Spring Harbor Laboratory Press, NY, N.Y.]. Other vectors and expression systems, including bacterial, yeast, and insect cell systems, can be used but are not preferred due to differences in, or lack of, glycosylation.

Recombinant factor VIII protein can be expressed in a variety of cells commonly used for culture and recombinant mammalian protein expression. In particular, a number of rodent cell lines have been found to be especially useful hosts for expression of large proteins. Preferred cell lines, available from the American Type Culture Collection, Rockville, MD, include baby hamster kidney cells, and Chinese hamster ovary (CHO) cells which are cultured using routine procedures and media.

The basis for the greater coagulant activity of porcine factor VIII appears to be the more rapid spontaneous dissociation of the human A2 subunit from human factor VIIIa than the porcine A2 subunit from porcine factor VIIIa. Dissociation of the A2 subunit leads to loss of activity, [Lollar, P. et al. (1990) J. Biol. Chem. 265:1688-1692; Lollar, P. et al. (1992) J. Biol. Chem. 267:23652-23657; Fay, P. J. et al. (1992) J. Biol. Chem. 267:13246-13250].

Factor VIII Molecules with Reduced Immunoreactivity

Epitopes that are immunoreactive with antibodies that inhibit the coagulant activity of factor VIII (“inhibitors” or “inhibitory antibodies”) have been characterized based on known structure-function relationships in factor VIII. Presumably, inhibitors could act by disrupting any of the macromolecular interactions associated with the domain structure of factor VIII or its associations with von Willebrand factor, thrombin, factor Xa, factor Ixa, or factor X. However, most inhibitory antibodies to human factor VIII act by binding to epitopes located in the 40 kDa A2 domain or 20 kDa C2 domain of factor VIII, disrupting specific functions associated with these domains, as described by Fulcher et al. (1985) Proc. Natl. Acad, Sci USA 82:7728-7732; and Scandella et al. (1988) Proc. Natl. Acad. Sci. USA 85:6152-6156. In addition to the A2 and C2 epitopes, there may be a third epitope in the A3 or C1 domain of the light chain of factor VIII, according to Scandella et al. (1993) Blood 82:1767-1775. The significance of this putative third epitope is unknown, but it appears to account for a minor fraction of the epitope reactivity in factor VIII.

Anti-A2 antibodies block factor X activation, as shown by Lollar et al. (1994) J. Clin. Invest. 93:2497-2504. Previous mapping studies by deletion mutagenesis described by Ware et al. (1992) Blood Coagul. Fibrinolysis 3:703-716, located the A2 epitope to within a 20 kDa region of the NH₂-terminal end of the 40 kDa A2 domain. Competition immunoradiometric assays have indicated that A2 inhibitors recognize either a common epitope or narrowly clustered epitopes, as described by Scandella et al. (1992) Throm. Haemostas. 67:665-671, and as demonstrated in U.S. Pat. No. 5,859,204.

Modified factor VIII molecules can be tested in humans for their reduced antigenicity and/or immunogenicity in clinical trials. In one type of trial, designed to determine whether the factor VIII is immunoreactive with inhibitory antibodies, factor VIII is administered, preferably by intravenous infusion, to approximately 25 patients having factor VIII deficiency who have antibodies that inhibit the coagulant activity of therapeutic human factor VIII. The dosage of the animal or modified animal factor VIII is in a range between 5 and 50 Units/kg body weight, preferably 10-50 Units/kg, and most preferably 40 Units/kg body weight. Approximately 1 hour after each administration, the recovery of factor VIII from blood samples is measured in a one-stage coagulation assay. Samples are taken again approximately 5 hours after infusion, and recovery is measured. Total recovery and the rate of disappearance of factor VIII from the samples is predictive of the antibody titer and inhibitory activity. If the antibody titer is high, factor VIII recovery usually cannot be measured. The recovery results are compared to the recovery results in patients treated with plasma-derived human factor VIII, recombinant human factor VIII, plasma-derived porcine factor VIII, and other commonly used therapeutic forms of factor VIII or factor VIII substitutes.

After identification of clinically significant epitopes, recombinant factor VIII molecules can be expressed that have less than or equal cross-reactivity compared with plasma-derived porcine factor VIII when tested in vitro against a broad survey of inhibitor plasmas. Additional mutagenesis in epitopic regions can be done to reduce cross-reactivity. Reduced cross-reactivity, although desirable, is not necessary to produce a product that may have advantages over the existing plasma-derived porcine factor VIII concentrate, which can produce side effects due to contaminant porcine proteins or contaminant infectious agents such as viruses or prions. A recombinant porcine or modified porcine factor VIII molecule will not contain foreign porcine proteins.

Diagnostic Assays

The factor VIII CDNA and/or protein expressed therefrom, in whole or in part, can be used in assays as diagnostic reagents for the detection of inhibitory antibodies to human or animal factor VIII or modified animal VIII in substrates, including, for example, samples of serum and body fluids of human patients with factor VIII deficiency. These antibody assays include assays such as ELISA assays, immunoblots, radioimmunoassays, immunodiffusion assays, and assay of factor VIII biological activity (e.g., by coagulation assay). Techniques for preparing these reagents and methods for use thereof are known to those skilled in the art. For example, an immunoassay for detection of inhibitory antibodies in a patient serum sample can include reacting the test sample with a sufficient amount of the factor VIII such that a detectable complex can be formed with the inhibitory antibodies in the sample.

Nucleic acid and amino acid probes can be prepared based on the sequence of the modified factor VIII cDNA or protein molecule or fragments thereof. In some embodiments, these can be labeled using dyes or enzymatic, fluorescent, chemiluminescent, or radioactive labels that are commercially available. The amino acid probes can be used, for example, to screen sera or other body fluids where the presence of inhibitors to human, animal, or hybrid human/animal factor VIII is suspected. Levels of inhibitors can be quantitated in patients and compared to healthy controls, and can be used, for example, to determine whether a patient with a factor VIII deficiency can be treated with an animal or modified animal factor VIII. The cDNA probes can be used, for example, for research purposes in screening DNA libraries.

Preparation of Recombinant Factor VIII

Recombinant factor VIII can be produced through the use of eukaryotic protein expression systems. In general, an eukaryotic cell line, which is deficient in a required gene, is transformed with a vector comprising the gene that it has a deficiency for, and the recombinant DNA which one wishes to express. Transformation can be accomplished by techniques such as electroporation or viral delivery. The cell line chosen to produce the protein is selected to be compatible with the protein of interest, capable of continuously expressing the protein of interest, capable of growing on a medium which facilitates purification of the protein of interest, along with other factors known to those skilled in the art. Examples of such techniques are disclosed in European Patent Application 0 302 968 A2 and U.S. Pat. No. 5,149,637, both of which are incorporated by reference in their entirety.

Testing of Recombinant Factor VIII Molecules

The recombinant factor VIII molecules can be tested in humans for their reduced antigenicity and/or immunogenicity in at least two types of clinical trials. In one type of trial, designed to determine whether the recombinant or recombinant hybrid factor VIII is immunoreactive with inhibitory antibodies, recombinant or recombinant hybrid factor VIII is administered, preferably by intravenous infusion, to approximately 25 patients having factor VIII deficiency who have antibodies to factor VIII that inhibit the coagulant activity of therapeutic human or porcine factor VIII. The dosage of the recombinant or recombinant hybrid factor VIII is in a range between 5 and 50 Units/kg body weight, preferably 10-50 Units/kg, and most preferably 40 Units/kg body weight. Approximately 1 hour after each administration, the recovery of factor VIII from blood samples is measured in a one-stage coagulation assay. Samples are taken again approximately 5 hours after infusion, and recovery is measured. Total recovery and the rate of disappearance of factor VIII from the samples is predictive of the antibody titer and inhibitory activity. If the antibody titer is high, factor VIII recovery usually cannot be measured. The recovery results are compared to the recovery results in patients treated with plasma-derived human factor VIII, recombinant human factor VIII, porcine factor VIII, and other commonly used therapeutic forms of factor VIII or factor VIII substitutes.

In a second type of clinical trial, designed to determine whether the recombinant or recombinant hybrid factor VIII is immunogenic, i.e., whether patients will develop inhibitory antibodies, recombinant or recombinant hybrid factor VIII is administered, as described in the preceding paragraph, to approximately 100 previously untreated hemophiliac patients who have not developed antibodies to factor VIII. Treatments are given approximately every 2 weeks over a period of 6 months to 1 year. At 1 to 3 month intervals during this period, blood samples are drawn and Bethesda assays or other antibody assays are performed to determine the presence of inhibitory antibodies. Recovery assays can also be done, as described above, after each infusion. Results are compared to hemophiliac patients who receive plasma-derived human factor VIII, recombinant human factor VIII, porcine factor VIII, or other commonly used therapeutic forms of factor VIII or factor VIII substitutes.

Pharmaceutical Compositions

Pharmaceutical compositions comprising recombinant or recombinant hybrid (or modified) factor VIII, alone or in combination with appropriate pharmaceutical stabilization compounds, delivery vehicles, and/or carrier vehicles, are prepared according to known methods, as described in Remington's Pharmaceutical Sciences by E. W. Martin.

In one preferred embodiment, the preferred carriers or delivery vehicles for intravenous infusion are physiological saline or phosphate buffered saline.

In another preferred embodiment, suitable stabilization compounds, delivery vehicles, and carrier vehicles include but are not limited to other human or animal proteins such as albumin.

Phospholipid vesicles or liposomal suspensions are also preferred as pharmaceutically acceptable carriers or delivery vehicles. These can be prepared according to methods known to those skilled in the art and can contain, for example, phosphatidylserine/phosphatidylcholine or other compositions of phospholipids or detergents that together impart a negative charge to the surface, since factor VIII binds to negatively charged phospholipid membranes. Liposomes may be prepared by dissolving appropriate lipid(s) (such as stearoyl phosphatidyl ethanolamine, stearoyl phosphatidyl choline, arachidoyl phosphatidyl choline, and cholesterol) in an inorganic solvent that is then evaporated, leaving behind a thin film of dried lipid on the surface of the container. An aqueous solution of the hybrid factor VIII is then introduced into the container. The container is then swirled by hand to free lipid material from the sides of the container and to disperse lipid aggregates, thereby forming the liposomal suspension.

Recombinant or recombinant hybrid (or modified) factor VIII can be combined with other suitable stabilization compounds, delivery vehicles, and/or carrier vehicles, including vitamin K dependent clotting factors, tissue factor, and von Willebrand factor (vWf) or a fragment of vWf that contains the factor VIII binding site, and polysaccharides such as sucrose.

Recombinant or recombinant hybrid (or modified) factor VIII can also be delivered by gene therapy in the same way that human factor VIII can be delivered, using delivery means such as retroviral vectors. This method consists of incorporation of factor VIII cDNA into human cells that are transplanted directly into a factor VIII deficient patient or that are placed in an implantable device, permeable to the factor VIII molecules but impermeable to cells, that is then transplanted. The preferred method will be retroviral-mediated gene transfer. In this method, an exogenous gene (e.g., a factor VIII cDNA) is cloned into the genome of a modified retrovirus. The gene is inserted into the genome of the host cell by viral machinery where it will be expressed by the cell. The retroviral vector is modified so that it will not produce virus, preventing viral infection of the host. The general principles for this type of therapy are known to those skilled in the art and have been reviewed in the literature [e.g., Kohn, D. B. et al. (1989) Transfusion 29:812-820].

Recombinant or recombinant hybrid (or modified) factor VIII can be stored bound to vWf to increase the half-life and shelf-life of the hybrid molecule. Additionally, lyophilization of factor VIII can improve the yields of active molecules in the presence of vWf. Current methods for storage of human and animal factor VIII used by commercial suppliers can be employed for storage of hybrid or modified factor VIII. These methods include: (1) lyophilization of factor VIII in a partially-purified state (as a factor VIII “concentrate” that is infused without further purification); (2) immunoaffinity-purification of factor VIII by the Zimmerman method and lyophilization in the presence of albumin, which stabilizes the factor VIII; (3) lyophilization of recombinant factor VIII in the presence of albumin.

Additionally, hybrid factor VIII has been indefinitely stable at 40° C. in 0.6 M NaCl, 20 mM MES, and 5 mM CaCl₂ at pH 6.0 and also can be stored frozen in these buffers and thawed with minimal loss of activity.

Methods of Treatment

Recombinant or recombinant hybrid (or modified) factor VIII is used to treat uncontrolled bleeding due to factor VIII deficiency (e.g., intraarticular, intracranial, or gastrointestinal hemorrhage) in hemophiliacs with and without inhibitory antibodies and in patients with acquired factor VIII deficiency due to the development of inhibitory antibodies. The active materials are preferably administered intravenously.

Additionally, recombinant or recombinant hybrid factor VIII can be administered by transplant of cells genetically engineered to produce the hybrid or by implantation of a device containing such cells, as described above.

In a preferred embodiment, pharmaceutical compositions of recombinant or recombinant hybrid (or modified) factor VIII alone or in combination with stabilizers, delivery vehicles, and/or carriers are infused into patients intravenously according to the same procedure that is used for infusion of human or animal factor VIII.

The treatment dosages of recombinant or recombinant hybrid (or modified) factor VIII composition that must be administered to a patient in need of such treatment will vary depending on the severity of the factor VIII deficiency. Generally, dosage level is adjusted in frequency, duration, and units in keeping with the severity and duration of each patient's bleeding episode. Accordingly, the hybrid factor VIII is included in the pharmaceutically acceptable carrier, delivery vehicle, or stabilizer in an amount sufficient to deliver to a patient a therapeutically effective amount of the hybrid to stop bleeding, as measured by standard clotting assays.

Factor VIII is classically defined as that substance present in normal blood plasma that corrects the clotting defect in plasma derived from individuals with hemophilia A. The coagulant activity in vitro of purified and partially-purified forms of factor VIII is used to calculate the dose of factor VIII for infusions in human patients and is a reliable indicator of activity recovered from patient plasma and of correction of the in vivo bleeding defect. There are no reported discrepancies between standard assay of novel factor VIII molecules in vitro and their behavior in the dog infusion model or in human patients, according to: Lusher, J. M. et al. 328 New Engl. J Med. 328:453459; Pittman, D. D. et al. (1992) Blood 79:389-397; and Brinkhous et al. (1985) Proc. Natl. Acad. Sci. 82:8752-8755.

Usually, the desired plasma factor VIII level to be achieved in the patient through administration of the recombinant or recombinant hybrid factor VIII is in the range of 30-100% of normal. In a preferred mode of administration of the recombinant or recombinant hybrid factor VIII, the composition is given intravenously at a preferred dosage in the range from about 5 to 50 units/kg body weight, more preferably in a range of 10-50 units/kg body weight, and most preferably at a dosage of 20-40 units/kg body weight; the interval frequency is in the range from about 8 to 24 hours (in severely affected hemophiliacs); and the duration of treatment in days is in the range from 1 to 10 days or until the bleeding episode is resolved. See, e.g., Roberts, H. R., and M. R. Jones, “Hemophilia and Related Conditions—Congenital Deficiencies of Prothrombin (Factor II, Factor V, and Factors VIII to XII),” Ch. 153, 1453-1474, 1460, in Hematology, Williams, W. J., et al. ed. (1990). Patients with inhibitors may require more recombinant or recombinant hybrid factor VIII or patients may require less recombinant or recombinant hybrid factor VIII because of its higher specific activity than human factor VIII or decreased antibody reactivity or immunogenicity. As in treatment with human or porcine factor VIII, the amount of recombinant or recombinant hybrid factor VIII infused is defined by the one-stage factor VIII coagulation assay and, in selected instances, in vivo recovery is determined by measuring the factor VIII in the patient's plasma after infusion. It is to be understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

Treatment can take the form of a single intravenous administration of the composition or periodic or continuous administration over an extended period of time, as required. Alternatively, recombinant or recombinant hybrid factor VIII can be administered subcutaneously or orally with liposomes in one or several doses at varying intervals of time.

Factor VIII can also be used to treat uncontrolled bleeding due to factor VIII deficiency in hemophiliacs who have developed antibodies to human factor VIII. In this case, coagulant activity that is superior to that of human or animal factor VIII alone is not necessary. Coagulant activity that is inferior to that of human factor VIII (i.e., less than 3,000 units/mg) will be useful if that activity is not neutralized by antibodies in the patient's plasma.

The recombinant or recombinant hybrid (or modified) factor VIII molecule and the methods for isolation, characterization, making, and using it generally described above will be further understood with reference to the following non-limiting examples.

### EXAMPLES

Materials—Citrated hemophilia A plasma and normal pooled human plasma (FACT) were purchased from George King Biomedical, Inc. (Overland Park, Kans.). Heparin-Sepharose was purchased from Sigma Chemical Co.(St. Louis, Mo.). Fetal bovine serum, geneticin, penicillin, streptomycin, DMEMJF12 medium and AIM-V medium were purchased from Life Technologies, Inc. (Gaithersburg, Md.). HP20, a B-domainless hybrid human/porcine fVIII molecule containing human A1, A2, ap-A3, and C1 domains and the porcine C2 domain was prepared as described previously [Healey, J. F., (1998) supra].

Plasmid DNA was purified using a Qiagen Plasmid Maxi Kit (Qiagen, Inc., Valencia, Calif.). PCR reactions were done using a Hybrid OmniGene thermocycler using Pfu DNA polymerase. PCR products were gel purified, precipitated with ethanol, and ligated into plasmid DNA using T4 DNA ligase (Rapid DNA Ligation Kit, Boehringer Mannheim, Indianapolis, Ind.). Insert-containing plasmids were used to transform E. coli Epicurean XL1-Blue cells. All novel fVIII DNA sequences generated by PCR were confirmed by dideoxy sequencing using an Applied Biosystems (Foster City, Calif.) 373a automated DNA sequencer and the PRISM dye terminator kit.

### EXAMPLE 1

### Construction of fVIII Mutant cDNAs

Transfected dell lines were maintained in Dulbecco's modified Eagle's medium-F12 containing 10% fetal bovine serum, 50 U/ml penicillin, and 50 μg/ml streptomycin. Fetal bovine serum was heat inactivated for one hour at 56° C. before use. Mutant cDNAs in ReNeo were stably transfected into BHK cells, selected for geneticin resistance, switched to serum-free, AIM-V medium for expression, and partially purified by heparin-Sepharose chromatography as described previously [Healey, J. F. et al (1998) supra].

### EXAMPLE 2

### FVIII and fVIII Inhibitor Assays

The activity of recombinant fVIII proteins was measured by one-stage clotting assay [Bowie, E. J. W. and Owen, Calif. (1984) In Disorders of Hemostasis, O. D. Ratnoff and C. D. Forbes, editors. Grune & Stratton, Inc., Orlando, Fla. 43-72]. One unit of fVIII is defined as the activity in one ml of normal citrated human plasma. FVIII inhibitor titers were measured by a modification of the Bethesda assay [Kasper, C. K. et al. (1975) Thromb. Diath. Haemorrh. 34:869-872] as follows. Recombinant fVIII was added to hemophilia A plasma to a final concentration of 0.8-1.2 units per ml and incubated with varying concentrations of inhibitor for 2 hours at 37° C. To determine the 50% inhibition point that defines the Bethesda unit, dilutions of inhibitor were made that produced residual activities that spanned at least the 35% to 65% range. In some cases, replicate dilutions were made, in which case the average was used. An average of 10 dilutions was made for the determination of each Bethesda titer. The data were fitted by nonlinear regression using the Marquardt algorithm (SigmaPlot 5.0, SPSS, Inc.) to the equation

% Residual activity =m (log x−log x₅₀)+50

where the fitted parameter x₅₀ is the reciprocal dilution that produces 50% inhibition, the fitted parameter m is the slope of the semi-log line and the independent variable x is the reciprocal dilution of the inhibitor sample.

The Bethesda titer equals x₅₀⁻¹. The estimate of the standard error (SD) of the Bethesda titer was calculated by multiplying the Bethesda titer by the coefficient of variation of x₅₀. The Bethesda titers of fVIII molecules were compared by Student's t test. The mass concentration of fVIII in partially purified preparations was determined by a sandwich ELISA using ESH4 as capture antibody and biotinylated ESH8 as detection antibody as described previously [Lubin, I. M. et al. (1994) 269:8639-8641]. Samples were assayed in quadruplicate.

### Example 3

### Bethesda Titers of C2-Specific Plasmas against C2 Mutants

fVIII inhibitor plasmas designated DR, EE, EEE, JF, LK, NF, JM, and WC were tested against C2 mutants Y2195H, Y2195A, F2196L, F2196A, R2215K, R2215A, R2220K, R2220A, F2290S, F2290A, W2313F, and R2320A and were compared to HSQ, see table 1. Reduction in antigenicity was seen in the DR and JF plasmas, but not the other six plasmas. DR recognizes R2215, R2220 and F2196 strongly, whereas JF recognizes R2215 strongly.

TABLE 1
Bethesda titers of patient C2-specific plasmas against human B-
domainless fVIII C2 mutants (% of HSQ titer)

| | DR | EE | EEE | JF | LK | NF | JM | WC |
|---|---|---|---|---|---|---|---|---|
| HSQ | 100 | 100 | 100 | 100 | 100 | 100 | 100 | 100 |
| R2215A | 8 | 140 | 62 | 7 | 70 | 81 | 91 | 68 |
| R2215K | 91 | 152 | 68 | 102 | 68 | 95 | 85 | 112 |
| R2220A | 4 | 119 | 58 | 41 | 85 | 129 | 123 | 121 |
| R2220K | 4 | 130 | 54 | 107 | 79 | 75 | 113 | 78 |
| W2313F | 41 | 138 | 99 | 133 | 50 | 80 | 93 | 89 |
| R2320A | 87 | 117 | 80 | 90 | 89 | 185 | 139 | 118 |
| G2290S | 74 | 130 | 129 | 70 | 118 | 162 | 123 | 159 |
| F2290A | 64 | 131 | 103 | 73 | 57 | 97 | 99 | 119 |
| Y2195H | 69 | 93 | 60 | 63 | 65 | 71 | 86 | 105 |
| Y2195A | 37 | 131 | 116 | 112 | 74 | 113 | 125 | 92 |
| F2169L | 14 | 74 | 99 | 72 | 89 | 155 | 89 | 99 |
| F2196A | 8 | 143 | 84 | 172 | 68 | 99 | 122 | 69 |

1. A modified human factor VIII comprising at least one amino acid substitution in the C2 domain wherein the substitution is limited to at least one position selected from the group consisting of 2215, 2313, 2220, 2320, 2195, 2196 and 2290 corresponding to SEQ ID NO:2.

2. The modified human factor VIII of claim 1 lacking a B-domain.

3. The modified human factor VIII of claim 12 wherein the substitution is alanine or lysine substituted for arginine 2215 corresponding to SEQ ID NO:2.

4. The modified human factor VIII of claim 13 wherein the substitution is alanine or lysine substituted for arginine 2220 corresponding to SEQ ID NO:2.

5. The modified human factor VIII of claim 1 wherein one of the substitutions is phenylalanine substituted for tryptophan 2313 corresponding to SEQ ID NO:2.

6. The modified human factor VIII of claim 1 wherein one of the substitutions is alanine substituted for arginine 2320 corresponding to SEQ ID NO:2.

7. The modified human factor VIII of claim 1 wherein one of the substitutions is alanine or serine substituted for phenylalanine 2290 corresponding to SEQ ID NO:2.

8. The modified human factor VIII of claim 1 wherein one of the substitutions is histidine or alanine substituted for tyrosine 2195 corresponding to SEQ ID NO:2.

9. The modified human factor VIII of claim 14 wherein the substitution is leucine or alanine substituted for phenylalanine 2196 corresponding to SEQ ID NO:2.

10-11 (Canceled)

12. The modified human factor VIII of claim 1 wherein one of the substitutions is at position 2215 corresponding to SEQ ID NO:2.

13. The modified human factor VIII of claim 1 wherein one of the substitutions is at position 2220 corresponding to SEQ ID NO:2.

14. The modified human factor VIII of claim 1 wherein one of the substitutions is at position 2196 corresponding to SEQ ID NO:2.

15-17 (Canceled)

18. The modified human factor VIII of claim 1 which has reduced antigenicity as compared to the corresponding human protein.

19. The modified human factor VIII of claim 1 which has reduced immunogenicity as compared to the corresponding human protein.

20. The modified human factor VIII of claim 1 which has reduced immunogenicity and reduced antigenicity as compared to the corresponding human protein.

21. The modified human factor VIII of claim 1 which has a specific activity greater than about 2,000 units per milligram.

22. The modified human factor VIII of claim 21 which has a specific activity greater than about 3,000 units per milligram.

23. The modified human factor VIII of claim 22 which has a specific activity greater than about 5,000 units per milligram.

24. The modified human factor VIII of claim 23 which has a specific activity greater than about 10,000 units per milligram.

25. The modified human factor VIII of claim 1 which is a single mutant.

26. The modified human factor VIII of claim 1 which is a double mutant.

27. The modified human factor VIII of claim 1 which is a triple mutant.

28. The modified human factor VIII of claim 1 which is a quadruple mutant.

29. The modified human factor VIII of claim 1 which has lower antigenicity towards at least one C2-specific inhibitory antibody as compared to the corresponding human factor VIII.

30. The modified human factor VIII of claim 1 which has an increased or decreased Bethesda titer towards at least one inhibitory antibody preparation as compared to the corresponding human factor VIII or recombinant factor VIII comprising the amino acid sequence of the corresponding human factor VIII.

31-36 (Canceled)

37. A method for modifying human factor VIII such that reactivity to an inhibitory antibody is reduced and procoagulant activity is retained comprising substituting an immuno-reactivity reducing amino acid for the naturally occurring amino acid in the C2 domain wherein the substitution is limited to at least one of the amino acids selected from the group consisting of 2215, 2313, 2220,2320,2195,2196 and 2290 corresponding to SEQ ID NO:2.

38. The method of claim 37 wherein at least one of the substitutions is at position 2215 corresponding to SEQ ID NO:2.

39. The method of claim 37 wherein at least one of the substitutions is at position 2313 corresponding to SEQ ID NO:2.

40. The method of claim 37 wherein at least one of the substitutions is at position 2220 corresponding to SEQ ID NO:2.

41. The method of claim 37 wherein at least one of the substitutions is at position 2320 corresponding to SEQ ID NO:2.

42. The method of claim 37 wherein at least one of the substitutions is at position 2195 corresponding to SEQ ID NO:2.

43. The method of claim 37 wherein at least one of the substitutions is at position 2196 corresponding to SEQ ID NO:2.

44. The method of claim 37 wherein the modified human factor VIII is a single mutant.

45. The method of claim 37 wherein the modified human factor VIII is a double mutant.

46. The method of claim 37 wherein the modified human factor VIII is a triple mutant.

47. The method of claim 37 wherein the modified human factor VIII is a quadruple mutant.

48. A method for modifying human factor VIII such that antigenicity is reduced and procoagulant activity is retained comprising substituting an immunoreactivity reducing amino acid for the naturally occurring amino acid in the C2 domain wherein the substitution is limited to at least one of the amino acids selected from the group consisting of 2215, 2313, 2220, 2320, 2195, 2196 and 2290 corresponding to SEQ ID NO:2.

49. The method of claim 48 wherein at least one of the substitutions is at position 2215 corresponding to SEQ ID NO:2.

50. The method of claim 48 wherein at least one of the substitutions is at position 2213 corresponding to SEQ ID NO:2.

51. The method of claim 48 wherein at least one of the substitutions is at position 2220 corresponding to SEQ ID NO:2.

52. The method of claim 48 wherein at least one of the substitutions is at position 2320 corresponding to SEQ ID NO:2.

53. The method of claim 48 wherein at least one of the substitutions is at position 2195 corresponding to SEQ ID NO:2.

54. The method of claim 48 wherein at least one of the substitutions is at position 2196 corresponding to SEQ ID NO:2.

55. The method of claim 48 wherein at least one of the substitutions is at position 2290 corresponding to SEQ ID NO:2.

56. The method of claim 48 wherein the modified human factor VIII is a single mutant.

57. The method of claim 48 wherein the modified human factor VIII is a double mutant.

58. The method of claim 48 wherein the modified human factor VIII is a triple mutant.

59. The method of claim 48 wherein the modified human factor VIII is a quadruple mutant.

60. (Canceled) 61. An isolated nucleic acid molecule encoding a modified human factor VIII comprising an substitution in the C2 domain wherein is limited to at least one of the amino acids selected from the group consisting of 2215, 2313, 2220,2320, 2195,2196 and 2290 corresponding to SEQ ID NO:2.

62. An expression vector comprising the nucleic acid molecule of claim 61.

63. The nucleic acid molecule of claim 61 wherein the factor VIII lacks a B-domain.

64. The nucleic acid molecule of claim 61 wherein one of the substitutions is at position 2220 corresponding to SEQ ID NO:2.

65. The nucleic acid molecule of claim 61 wherein one of the substitutions is at position 2215 corresponding to SEQ ID NO:2.

66. The nucleic acid molecule of claim 61 wherein one of the substitutions is at position 2196 corresponding to SEQ ID NO:2.

67. A host cell transfected with the expression vector of claim 62.


SEQUENCE LISTING (7 sequences)

SEQ ID NO: 1 - DNA, 9009 bp, Homo sapiens
cagtgggtaagttccttaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccattggtcttaattctgaccaataaaaaaataagtcaggaggatgcaattgttgaaagctttgaaataaaataacatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgcaccccttcactatgactacacagaactctcctgatagtaaagggggctggaggcaaggataagttatagagcagttggaggaagcatccaaagactgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacataca

SEQ ID NO: 2 - protein, 2332 aa, Homo sapiens
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYEGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - DNA, 6402 bp, Porcine
  CDS (1)..(6399)
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtctcacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcggttgccaagaagcatcccaaaacctgggtgcactacatctctgcagaggaggaggactgggactacgcccccgcggtccccagccccagtgacagaagttataaaagtctctacttgaacagtggtcctcagcgaattggtaggaaatacaaaaaagctcgattcgtcgcttacacggatgtaacatttaagactcgtaaagctattccgtatgaatcaggaatcctgggacctttactttatggagaagttggagacacacttttgattatatttaagaataaagcgagccgaccatataacatctaccctcatggaatcactgatgtcagcgctttgcacccagggagacttctaaaaggttggaaacatttgaaagacatgccaattctgccaggagagactttcaagtataaatggacagtgactgtggaagatgggccaaccaagtccgatcctcggtgcctgacccgctactactcgagctccattaatctagagaaagatctggcttcgggactcattggccctctcctcatctgctacaaagaatctgtagaccaaagaggaaaccagatgatgtcagacaagagaaacgtcatcctgttttctgtattcgatgagaatcaaagctggtacctcgcagagaatattcagcgcttcctccccaatccggatggattacagccccaggatccagagttccaagcttctaacatcatgcacagcatcaatggctatgtttttgatagcttgcagctgtcggtttgtttgcacgaggtggcatactggtacattctaagtgttggagcacagacggacttcctctccgtcttcttctctggctacaccttcaaacacaaaatggtctatgaagacacactcaccctgttccccttctcaggagaaacggtcttcatgtcaatggaaaacccaggtctctgggtcctagggtgccacaactcagacttgcggaacagagggatgacagccttactgaaggtgtatagttgtgacagggacattggtgattattatgacaacacttatgaagatattccaggcttcttgctgagtggaaagaatgtcattgaacccagaagctttgcccagaattcaagaccccctagtgcgagccaaaagcaattccaaaccatcacaagtccagaagatgacgtggagcttgacccgcagtctggagagagaacccaagcactggaagaactaagtgtcccctctggtgatgggtcgatgctcttgggacagaatcctgctccacatggctcatcctcatctgatcttcaagaagccaggaatgaggctgatgattatttacctggagcaagagaaagaaacacggccccatccgcagcggcacgtctcagaccagagctgcatcacagtgccgaaagagtacttactcctgagccagagaaagagttgaagaaacttgattcaaaaatgtctagttcatcagaccttctaaagacttcgccaacaattccatcagacacgttgtcagcggagactgaaaggacacattccttaggccccccacacccgcaggttaatttcaggagtcaattaggtgccattgtacttggcaaaaattcatctcactttattggggctggtgtccctttgggctcgactgaggaggatcatgaaagctccctgggagaaaatgtatcaccagtggagagtgacgggatatttgaaaaggaaagagctcatggacctgcttcactgaccaaagacgatgttttatttaaagttaatatctctttggtaaagacaaacaaggcacgagtttacttaaaaactaatagaaagattcacattgatgacgcagctttattaactgagaatagggcatctgcaacgtttatggacaaaaatactacagcttcgggattaaatcatgtgtcaaattggataaaagggccccttggcaagaaccccctaagctcggagcgaggccccagtccagagcttctgacatcttcaggatcaggaaaatctgtgaaaggtcagagttctgggcaggggagaatacgggtggcagtggaagaggaagaactgagcaaaggcaaagagatgatgcttcccaacagcgagctcacctttctcactaactcggctgatgtccaaggaaacgatacacacagtcaaggaaaaaagtctcgggaagagatggaaaggagagaaaaattagtccaagaaaaagtcgacttgcctcaggtgtatacagcgactggaactaagaatttcctgagaaacatttttcaccaaagcactgagcccagtgtagaagggtttgatggggggtcacatgcgccggtgcctcaagacagcaggtcattaaatgattcggcagagagagcagagactcacatagcccatttctcagcaattagggaagaggcacccttggaagccccgggaaatcgaacaggtccaggtccgaggagtgcggttccccgccgcgttaagcagagcttgaaacagatcagactcccgctagaagaaataaagcctgaaaggggggtggttctgaatgccacctcaacccggtggtctgaaagcagtcctatcttacaaggagccaaaagaaataacctttctttacctttcctgaccttggaaatggccggaggtcaaggaaagatcagcgccctggggaaaagtgccgcaggcccgctggcgtccgggaagctggagaaggctgttctctcttcagcaggcttgtctgaagcatctggcaaagctgagtttcttcctaaagttcgagttcatcgggaagacctgttgcctcaaaaaaccagcaatgtttcttgcgcacacggggatctcggccaggagatcttcctgcagaaaacacggggacctgttaacctgaacaaagtaaatagacctggaaggactccctccaagcttctgggtcccccgatgcccaaagagtgggaatccctagagaagtcaccaaaaagcacagctctcaggacgaaagacatcatcagtttacccctggaccgtcacgaaagcaatcattcaatagcagcaaaaaatgaaggacaagccgagacccaaagagaagccgcctggacgaagcagggagggcctggaaggctgtgcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccacctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaaggagtgtcaggctccactgggaatggcttctggacgcattagagattttcagatcacagcttcaggacagtatggacagtgggccccaaagctggccagacttcattattccggatcaatcaatgcctggagcaccaaggatccccactcctggatcaaggtggatctgttggcaccaatgatcattcacggcatcatgacccagggtgcccgtcagaagttttccagcctctacatctcccagtttatcatcatgtacagtcttgacgggaggaactggcagagttaccgagggaattccacgggcaccttaatggtcttctttggcaatgtggacgcatctgggattaaacacaatatttttaaccctccgattgtggctcggtacatccgtttgcacccaacacattacagcatccgcagcactcttcgcatggagttgatgggctgtgatttaaacagttgcagcatgcccctgggaatgcagaataaagcgatatcagactcacagatcacggcctcctcccacctaagcaatatatttgccacctggtctccttcacaagcccgacttcacctccaggggcggacgaatgcctggcgaccccgggtgagcagcgcagaggagtggctgcaggtggacctgcagaagacggtgaaggtcacaggcatcaccacccagggcgtgaagtccctgctcagcagcatgtatgtgaaggagttcctcgtgtccagtagtcaggacggccgccgctggaccctgtttcttcaggacggccacacgaaggtttttcagggcaatcaggactcctccacccccgtggtgaacgctctggaccccccgctgttcacgcgctacctgaggatccaccccacgagctgggcgcagcacatcgccctgaggctcgaggttctaggatgtgaggcacaggatctctactga

SEQ ID NO: 4 - protein, 2133 aa, Porcine
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYISAEEEDWDYAPAVPSPSDRSYKSLYLNSGPQRIGRKYKKARFVAYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTYEDIPGFLLSGKNVIEPRSFAQNSRPPSASQKQFQTITSPEDDVELDPQSGERTQALEELSVPSGDGSMLLGQNPAPHGSSSSDLQEARNEADDYLPGARERNTAPSAAARLRPELHHSAERVLTPEPEKELKKLDSKMSSSSDLLKTSPTIPSDTLSAETERTHSLGPPHPQVNFRSQLGAIVLGKNSSHFIGAGVPLGSTEEDHESSLGENVSPVESDGIFEKERAHGPASLTKDDVLFKVNISLVKTNKARVYLKTNRKIHIDDAALLTENRASATFMDKNTTASGLNHVSNWIKGPLGKNPLSSERGPSPELLTSSGSGKSVKGQSSGQGRIRVAVEEEELSKGKEMMLPNSELTFLTNSADVQGNDTHSQGKKSREEMERREKLVQEKVDLPQVYTATGTKNFLRNIFHQSTEPSVEGFDGGSHAPVPQDSRSLNDSAERAETHIAHFSAIREEAPLEAPGNRTGPGPRSAVPRRVKQSLKQIRLPLEEIKPERGVVLNATSTRWSESSPILQGAKRNNLSLPFLTLEMAGGQGKISALGKSAAGPLASGKLEKAVLSSAGLSEASGKAEFLPKVRVHREDLLPQKTSNVSCAHGDLGQEIFLQKTRGPVNLNKVNRPGRTPSKLLGPPMPKEWESLEKSPKSTALRTKDIISLPLDRHESNHSIAAKNEGQAETQREAAWTKQGGPGRLCAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKECQAPLGMASGRIRDFQITASGQYGQWAPKLARLHYSGSINAWSTKDPHSWIKVDLLAPMIIHGIMTQGARQKFSSLYISQFIIMYSLDGRNWQSYRGNSTGTLMVFFGNVDASGIKHNIFNPPIVARYIRLHPTHYSIRSTLRMELMGCDLNSCSMPLGMQNKAISDSQITASSHLSNIFATWSPSQARLHLQGRTNAWRPRVSSAEEWLQVDLQKTVKVTGITTQGVKSLLSSMYVKEFLVSSSQDGRRWTLFLQDGHTKVFQGNQDSSTPVVNALDPPLFTRYLRIHPTSWAQHIALRLEVLGCEAQDLY

SEQ ID NO: 5 - DNA, 7493 bp, Mus musculus
tctagagtttctttgctacaggtaccaaggaacagtcttttagaataggctaggaatttaaatacacctgaacgcccctcctcagtattctgttccttttcttaaggattcaaacttgttaggatgcacccagcaggaaatgggttaagccttagctcagccactcttcctattccagttttcctgtgcctgcttcctactacccaaaaggaagtaatccttcagatctgttttgtgctaatgctactttcactcacagtagataaacttccagaaaatcctctgcaaaatatttaggactttttactaaatcattacatttctttttgttcttaaaagctaaagttattttagagaagagttaaattttcatttctttagttgaacattttctagtaataaaagccatgcaaatagcactcttcgcttgcttctttctgagccttttcaatttctgctctagtgccatcagaagatactaccttggtgcagtggaattgtcctggaactatattcagagtgatctgctcagtgtgctgcatacagactcaagatttcttcctagaatgtcaacatcttttccattcaacacctccatcatgtataaaaagactgtgtttgtagagtacaaggaccagcttttcaacattgccaagcccaggccaccctggatgggtttgctaggtcctaccatttggactgaggttcatgacacagtggtcattacacttaaaaacatggcttctcatcctgtcagtcttcatgctgttggtgtgtcctactggaaagcttctgagggagatgaatatgaagatcagacaagccaaatggagaaggaagatgataaagttttccctggtgaaagtcatacttatgtttggcaagtcctgaaagagaatggtccaatggcctctgaccctccatgtctcacttactcatatatgtctcatgtggatctggtgaaagatttgaattcaggcctcattggagctctgctagtatgtaaagaaggcagtctctccaaagaaagaacacagatgttgtaccaatttgtactgctttttgctgtatttgatgaagggaagagctggcactcagaaacaaacgactcttatacacagtctatggattctgcatctgctagagactggcctaaaatgcacacagtcaatggctatgtaaacaggtctcttccaggtctgattggatgccataggaaatcagtctactggcacgtgattggaatgggcaccactcctgaaatacactcaatattcctcgaaggtcacacattttttgtgaggaaccaccgtcaagcttcattggagatatcaccaataactttccttactgctcaaacactcttgatagatcttgggcagttcctactattttgtcatatctcttcccataaacatgatggcatggaagcttatgtcaaagtagatagctgccctgaggaatcccaatggcaaaagaaaaataataatgaggaaatggaagattatgatgatgatctttattcagaaatggatatgttcacattggattatgacagctctccttttatccaaattcgctcggttgctaaaaagtaccctaaaacttggatacattatatttctgctgaggaggaagactgggactatgcaccttcagttcctacctcggataatggaagttataaaagccagtatctgagcaatggtcctcatcggattggtaggaaatataaaaaagtcagatttatagcatacacagatgaaacctttaagactcgtgaaactattcagcatgaatcaggactcttgggacctttactttatggagaagttggagacacactgttgattatttttaagaatcaagcaagccgaccatataacatttaccctcatggaatcactgatgtcagtcctctacatgcaaggagattgccaagaggtataaagcacgtgaaggatttgccaattcatccaggagagatattcaagtacaagtggacagttacagtagaagatggaccaactaaatcagatccacggtgcctgacccgctattattcaagtttcattaaccctgagagagatctagcttcaggactgattggccctcttctcatctgctacaaagaatctgtagatcaaaggggaaaccagatgatgtcagacaaaagaaatgtcatcctgttttctatatttgatgagaaccaaagctggtacatcacagagaacatgcaacgcttcctccccaatgcagctaaaacacagccccaggaccctgggttccaggcctccaacatcatgcacagcatcaatggctatgtttttgatagcttggagttgacagtttgtttgcatgaggtggcatactggcacattctcagtgttggagcacagacagacttcttatctatcttcttctctggatatactttcaaacacaaaatggtctatgaagatacacttaccctgttcccattctcaggagaaactgtctttatgtcgatggaaaacccaggtctatgggtcttggggtgtcataattcagactttcggaagagaggtatgacagcattgctgaaagtttctagttgtgacaagagcactagtgattattatgaagaaatatatgaagatattccaacacagttggtgaatgagaacaatgtcattgatcccagaagcttcttccagaatacaaatcatcctaatactaggaaaaagaaattcaaagattccacaattccaaaaaatgatatggagaagattgagcctcagtttgaagagatagcagagatgcttaaagtacagagtgtctcagttagtgacatgttgatgctcttgggacagagtcatcctactccacatggcttatttttatcagatggccaagaagccatctatgaggctattcatgatgatcattcaccaaatgcaatagacagcaatgaaggcccatctaaagtgacccaactcaggccagaatcccatcacagtgagaaaatagtatttactcctcagcccggcctccagttaagatccaataaaagtttggagacaactatagaagtaaagtggaagaaacttggtttgcaagtttctagtttgccaagtaatctaatgactacaacaattctgtcagacaatttgaaagcaacttttgaaaagacagattcttcaggatttccagatatgccagttcactctagtagtaaattaagtactactgcatttggtaagaaagcatattcccttgttgggtctcatgtacctttaaacgcgagtgaagaaaatagtgattccaacatattggattcaactttaatgtatagtcaagaaagtttaccaagagataatatattatcaatagagaatgatagattactcagagagaagaggtttcatggaattgctttattgaccaaagataatactttattcaaagacaatgtctccttaatgaaaacaaacaaaacatataatcattcaacaactaatgaaaaactacacactgagagcccaacatcaattgagaatagtacaacagacttgcaagatgccatattaaaggtcaatagtgagattcaagaagtaacagctttgattcatgatggaacacttttaggcaaaaattctacatatttgagactaaaccatatgctaaatagaactacctcaacaaaaaataaagacatatttcatagaaaagatgaagatcctattccacaagatgaagagaatacaatcatgccattttccaagatgttgttcttgtcagaatcttcaaattggtttaaaaagaccaatggaaataattccttgaactctgagcaagaacatagtccaaagcaattagtatatttaatgtttaaaaaatatgtaaaaaatcaaagtttcttgtcagagaaaaataaagtcacagtagaacaggatggatttacaaagaacataggacttaaagacatggcttttccacataatatgagcatatttcttaccactttgtctaacgtacatgaaaatggtaggcacaatcaagaaaaaaatattcaggaagagatagagaaggaagcactaattgaagagaaagtagttttgccccaggtgcacgaagcaactggctctaagaatttcttgaaagacatattgatactaggcactaggcaaaatataagtttatatgaagtacatgtaccagtacttcaaaacatcacatcaataaacaattcaacaaatacagtacagattcacatggagcatttctttaaaagaaggaaggacaaggaaacaaattcagaaggcttggtaaataaaaccagagaaatggtaaaaaactatccaagccagaagaatattactactcaacgtagtaaacgggctttgggacaattcagactgtcaactcaatggcttaaaaccataaactgttcaacacagtgtatcattaaacagatagaccacagcaaggaaatgaaaaagttcattactaaatcttccttatcagattcttctgtgattaaaagcaccactcagacaaatagttctgactcacacattgtaaaaacatcagcatttccaccaatagatctcaaaaggagtccattccaaaacaaattttctcatgttcaagcatcatcctacatttatgactttaagacaaaaagttcaagaattcaagaaagcaataatttcttaaaagaaaccaaaataaataacccttctttagccattctaccatggaatatgttcatagatcaaggaaaatttacctccccagggaaaagtaacacaaactcagtcacatataagaaacgtgagaacattattttcttgaaaccaactttgcctgaagaatctggcaaaattgaattgcttcctcaagtttccattcaagaggaagaaattttacctacagaaactagccatggatctcctggacacttgaatctcatgaaagaggtctttcttcagaaaatacaggggcctactaaatggaataaagcaaagaggcatggagaaagtataaaaggtaaaacagagagctctaaaaatactcgctcaaaactgctaaatcatcatgcttgggattatcattatgctgcacagataccaaaagatatgtggaaatccaaagagaagtcaccagaaattatatccattaagcaagaggacaccattttgtctctgaggcctcatggaaacagtcattcaataggggcaaatgagaaacaaaattggcctcaaagagaaaccacttgggtaaagcaaggccaaactcaaaggacatgctctcaaatcccaccagtgttgaaacgacatcaaagggaacttagtgcttttcaatcagaacaagaagcaactgactatgatgatgccatcaccattgaaacaatcgaggattttgacatttacagtgaggacataaagcaaggtccccgcagctttcaacagaaaacaaggcactattttattgcagctgtggaacgactctgggactatgggatgagtacatctcatgttctacgaaataggtatcaaagtgacaatgtacctcagttcaagaaagtagttttccaggaatttactgatggctcctttagtcagcccttatatcgtggagaattaaatgaacacctggggttgttgggcccatatataagagcagaagttgaagacaacattatggtaactttcaaaaaccaggcctcccgtccctactccttctattctagcctcatttcttataaagaagatcagagaggagaagaacctagaagaaactttgtcaagcctaatgaaaccaaaatttatttttggaaagtacaacatcatatggcacccacagaagatgagtttgactgcaaggcctgggcttatttctctgatgttgatcttgaaagagatatgcactcgggattaattggaccccttctgatttgccacgcgaacacactgaatcctgctcatgggagacaagtgtcagtacaggaatttgctctgcttttcactatctttgatgagaccaagagctggtacttcactgaaaacgtgaaaaggaactgcaagacaccctgcaatttccagatggaagaccccactttgaaagagaattatcgcttccatgcaatcaatggttatgtaatggataccctaccaggcttagtaatggctcaagatcaaaggattcgatggtatcttctcagcatgggcaacaatgagaacatccaatctattcatttcagtggacatgttttcactgtacggaaaaaagaggagtataaaatggcagtgtacaacctctacccaggtgtttttgagactctggaaatgataccatccagagctggaatatggcgagtagaatgccttattggcgagcacttacaggctgggatgagcactctttttctggtgtacagcaagcagtgtcagattcctcttggaatggcttctggaagcatccgtgatttccagattacagcttcaggacattatggacagtgggccccaaacctggcaagacttcattattccggatcaatcaatgcctggagtaccaaggagcccttttcttggatcaaggtagatctgttggcaccaatgattgttcatggcatcaagactcagggtgctcgtcagaaattttccagcctttatatctctcaatttatcatcatgtatagcctggatgggaagaagtggctgagttatcaaggaaattccactggaaccttaatggttttctttggcaatgtggactcatctgggattaagcataatagttttaatcctccaattattgctcgatatatccgtttgcaccccactcattctagcatccgtagtactcttcgcatggagttgatgggctgtgatttaaacagttgcagcataccattgggaatggaaagtaaagtaatatcagatacacaaatcactgcctcatcctacttcaccaacatgtttgctacttggtctccttcacaagctcgacttcacctccagggaaggactaatgcctggcgacctcaggtgaatgatccaaaacaatggttgcaagtggacttacaaaagacaatgaaagtcactggaataataacccagggagtgaaatctctctttaccagcatgtttgtgaaagagttccttatttccagcagtcaagatggccatcactggactcaaattttatacaatggcaaggtaaaggtttttcaggggaatcaggactcatccacacctatgatgaattctctagacccaccattactcactcgctatcttcgaattcacccccagatctgggagcaccaaattgctctgaggcttgagattctaggatgtgaggcccagcagcaatactgaggtagcctctgcatcacctgcttattccccttcctcagctcaaagattgtcttaatgttttattgctgtgaagagacactatgaccatggcaactctttataaaataaagcatttaatcagggctt

SEQ ID NO: 6 - protein, 2319 aa, Mus musculus
MQIALFACFFLSLFNFCSSAIRRYYLGAVELSWNYIQSDLLSVLHTDSRFLPRMSTSFPFNTSIMYKKTVFVEYKDQLFNIAKPRPPWMGLLGPTIWTEVHDTVVITLKNMASHPVSLHAVGVSYWKASEGDEYEDQTSQMEKEDDKVFPGESHTYVWQVLKENGPMASDPPCLTYSYMSHVDLVKDLNSGLIGALLVCKEGSLSKERTQMLYQFVLLFAVFDEGKSWHSETNDSYTQSMDSASARDWPKMHTVNGYVNRSLPGLIGCHRKSVYWHVIGMGTTPEIHSIFLEGHTFFVRNHRQASLEISPITFLTAQTLLIDLGQFLLFCHISSHKHDGMEAYVKVDSCPEESQWQKKNNNEEMEDYDDDLYSEMDMFTLDYDSSPFIQIRSVAKKYPKTWIHYISAEEEDWDYAPSVPTSDNGSYKSQYLSNGPHRIGRKYKKVRFIAYTDETFKTRETIQHESGLLGPLLYGEVGDTLLIIFKNQASRPYNIYPHGITDVSPLHARRLPRGIKHVKDLPIHPGEIFKYKWTVTVEDGPTKSDPRCLTRYYSSFINPERDLASGLIGPLLICYKESVDQRGNQMMSDKRNVILFSIFDENQSWYITENMQRFLPNAAKTQPQDPGFQASNIMHSINGYVFDSLELTVCLHEVAYWHILSVGAQTDFLSIFFSGYTFKHKMVYEDTLTLFPFSGETVFMSMENPGLWVLGCHNSDFRKRGMTALLKVSSCDKSTSDYYEEIYEDIPTQLVNENNVIDPRSFFQNTNHPNTRKKKFKDSTIPKNDMEKIEPQFEEIAEMLKVQSVSVSDMLMLLGQSHPTPHGLFLSDGQEAIYEAIHDDHSPNAIDSNEGPSKVTQLRPESHHSEKIVFTPQPGLQLRSNKSLETTIEVKWKKLGLQVSSLPSNLMTTTILSDNLKATFEKTDSSGFPDMPVHSSSKLSTTAFGKKAYSLVGSHVPLNASEENSDSNILDSTLMYSQESLPRDNILSIENDRLLREKRFHGIALLTKDNTLFKDNVSLMKTNKTYNHSTTNEKLHTESPTSIENSTTDLQDAILKVNSEIQEVTALIHDGTLLGKNSTYLRLNHMLNRTTSTKNKDIFHRKDEDPIPQDEENTIMPFSKMLFLSESSNWFKKTNGNNSLNSEQEHSPKQLVYLMFKKYVKNQSFLSEKNKVTVEQDGFTKNIGLKDMAFPHNMSIFLTTLSNVHENGRHNQEKNIQEEIEKEALIEEKVVLPQVHEATGSKNFLKDILILGTRQNISLYEVHVPVLQNITSINNSTNTVQIHMEHFFKRRKDKETNSEGLVNKTREMVKNYPSQKNITTQRSKRALGQFRLSTQWLKTINCSTQCIIKQIDHSKEMKKFITKSSLSDSSVIKSTTQTNSSDSHIVKTSAFPPIDLKRSPFQNKFSHVQASSYIYDFKTKSSRIQESNNFLKETKINNPSLAILPWNMFIDQGKFTSPGKSNTNSVTYKKRENIIFLKPTLPEESGKIELLPQVSIQEEEILPTETSHGSPGHLNLMKEVFLQKIQGPTKWNKAKRHGESIKGKTESSKNTRSKLLNHHAWDYHYAAQIPKDMWKSKEKSPEIISIKQEDTILSLRPHGNSHSIGANEKQNWPQRETTWVKQGQTQRTCSQIPPVLKRHQRELSAFQSEQEATDYDDAITIETIEDFDIYSEDIKQGPRSFQQKTRHYFIAAVERLWDYGMSTSHVLRNRYQSDNVPQFKKVVFQEFTDGSFSQPLYRGELNEHLGLLGPYIRAEVEDNIMVTFKNQASRPYSFYSSLISYKEDQRGEEPRRNFVKPNETKIYFWKVQHHMAPTEDEFDCKAWAYFSDVDLERDMHSGLIGPLLICHANTLNPAHGRQVSVQEFALLFTIFDETKSWYFTENVKRNCKTPCNFQMEDPTLKENYRFHAINGYVMDTLPGLVMAQDQRIRWYLLSMGNNENIQSIHFSGHVFTVRKKEEYKMAVYNLYPGVFETLEMIPSRAGIWRVECLIGEHLQAGMSTLFLVYSKQCQIPLGMASGSIRDFQITASGHYGQWAPNLARLHYSGSINAWSTKEPFSWIKVDLLAPMIVHGIKTQGARQKFSSLYISQFIIMYSLDGKKWLSYQGNSTGTLMVFFGNVDSSGIKHNSFNPPIIARYIRLHPTHSSIRSTLRMELMGCDLNSCSIPLGMESKVISDTQITASSYFTNMFATWSPSQARLHLQGRTNAWRPQVNDPKQWLQVDLQKTMKVTGIITQGVKSLFTSMFVKEFLISSSQDGHHWTQILYNGKVKVFQGNQDSSTPMMNSLDPPLLTRYLRIHPQIWEHQIALRLEILGCEAQQQY

SEQ ID NO: 7 - protein, 19 aa, Homo sapiens
  SIGNAL (1)..(19): Signal peptide of human factor VIII protein.
MQIELSTCFFLCLLRFCFS